(19) United States
(12) Patent Application Publication
(10) Pub. No.: US 2006/0257969 A1
(43) Pub. Date: Nov. 16, 2006

(54) Methods for determining contamination of fluid compositions

(76) Inventor: Peter Hug, Toledo, OH (US)

Correspondence Address: DUNLAP, CODDING & ROGERS P.C., PO BOX 16370, OKLAHOMA CITY, OK, 73113, US

(21) Appl. No.: 11/434,965

(22) Filed: May 16, 2006

Related U.S. Application Data
(60) Provisional application No. 60/681,510, filed on May 16, 2005.

(51) Int. Cl. C12Q 1/04

(52) U.S. Cl. 435/34

(57) ABSTRACT

Methods and systems for determining the contamination of a fluid composition are provided. The methods include both viability and species or genus determinations and are particularly well suited for use with metalworking fluid compositions. Methods of performing quality control services utilizing such methods, as well as kits that contain components utilized in such methods, are also provided.

### CROSS REFERENCE TO RELATED APPLICATIONS

This application claims benefit under 35 U.S.C. 119(e) of U.S. Ser. No. 60/681,510, filed May 16, 2005; the entire contents of which are hereby expressly incorporated herein by reference in their entirety.

### STATEMENT REGARDING FEDERALLY SPONSORED RESEARCH OR DEVELOPMENT

Not Applicable.

### FIELD OF THE INVENTION

The invention relates to methods and systems for determining microbial contamination of fluid compositions, such as metalworking fluid compositions.

### BACKGROUND OF THE INVENTION

Fluid compositions are employed in a variety of tasks and environments wherein knowledge of contaminants present in the fluid composition is important to insuring the proper and effective functioning and utilization of the fluid composition. Such fluid compositions for example are employed in cooling water tower systems, washing operations, machining processes, swimming pools, hydraulic fluids, plating operations and the like. Thus methods and devices for the simple direct determination of the presence of one or more contaminants in a fluid composition is important in the industrial arts.

In the shaping of a solid workpiece, such as for example a piece of metal, into a useful article, it is known to apply a cutting or non-cutting tool against the workpiece. This tool and/or the workpiece may be rotated with respect to each other, often at high speeds. Such high speeds are typically found in turning and grinding operations for shaping metals and other solid materials. In other cases the tool and workpiece are caused to have sliding contact with each other, such as in a punching operation. Still other shaping operations cause a tool to be applied against the workpiece with great force without cutting the workpiece, such as in metal rolling, drawing and ironing processes. High heat and friction are generated during these and other shaping methods, thus causing such problems as tool wear, distortion of the finished article, poor surface finish and out of tolerance dimensions for the article. High Scrap rates, tool wear and increased costs result from these problems. To overcome these and other problems it is known in the art to apply a metalworking fluid to the interface between the tool and the workpiece. The term “metalworking fluid” refers to a complex fluid composition applied to the interface between a tool and a metallic workpiece during the shaping of the workpiece by physical means. Such physical means are principally mechanical means exemplified by grinding, machining, turning, rolling, punching, extruding, spinning, drawing and ironing, stamping and forming, pressing and drilling operations, and the like.

Metalworking fluids (MWFs) are used throughout the manufacturing industry to provide a more efficient material removal or forming operation. Such fluids are selected generally for the purpose of cooling the workpiece and the tool during cutting operations, and to facilitate removal of chips during turning, grinding, and similar operations. MWFs are an important facet of many manufacturing operations in that they provide the required chip and heat removal properties necessary to achieve higher production outputs, increased tool life, and enhanced machined-surface finish and part quality.

The metalworking fluids applied to the interface between the tool and the workpiece in the metalworking art can be broadly classified into two categories. These categories are oils and aqueous based liquids or fluids. The oils are non-aqueous liquids comprising an oil or mixture of oils and one or more additives, such as for example, surfactants, extreme pressure agents, corrosion inhibitors, bactericides, fungicides and odor control agents. Aqueous based metalworking fluids are complex combinations of water, lubricant and additives. Many different lubricants are used in aqueous based metalworking fluids, and aqueous based metalworking fluids can be classified as soluble oils, synthetic fluids and semi synthetic fluids. The lubricants and many other components of aqueous based metalworking fluids are synthetic or naturally occurring organic compounds or mixtures of compounds. Lubricants used in the aqueous based metalworking fluids may include for example esters, amides, polyethers, amines and sulfonated oils. The lubricant component reduces friction between the tool and workpiece while the water helps dissipate the heat generated in the metalworking operation. Corrosion inhibitors are employed to reduce or prevent corrosion of the workpiece and the finished article as well as to reduce or prevent chemical attack on the tool. Bactericides and fungicides are used to reduce or prevent microbial or fungal attack on the constituents of the fluid, while the surfactant may be employed to form a stable suspension of water insoluble components in the water phase of the fluid. Thus, each component of the metalworking fluid has a function contributing to the overall utility and effectiveness of the metalworking fluid.

During its use, a metalworking fluid may undergo numerous changes, one of which is increased microbial growth, which results in microbial attack upon the lubricant and/or other components of the MWF composition. MWF's are susceptible to microbial attack by bacteria, fungi and/or yeasts (collectively termed herein “microbes”), because many of the components commonly used in MWF's are nutrients and growth promoters for many different kinds of bacteria, fungi and yeast. Microbial contamination of MWF's can cause one or more consequences such as but not limited to, odor development, decrease in pH, decrease in dissolved oxygen concentration, changes in emulsion stability (for water soluble oils and semisynthetic fluids), increased incidence of dermatitis, diseases such as but not limited to, hypersensitivity pneumonitis, workpiece surface-finish blemishes, clogged filters and lines, increased workpiece rejection rates, decreased tool life, and generally unpredictable changes in coolant chemistry. See, for example, Frederick J. Passman, “Microbial Problems in Metalworking Fluids,” Lubrication Engineering, pp. 431-3, May 1988; and I. Mattsby-Baltzer et al., “Microbial Growth and Accumulation in Industrial Metal-Working Fluids,” Applied and Environmental Microbiology, October 1989, pp. 2681-2689.

Among the bacteria that are commonly found in MWF's are aerobic bacteria, such as but not limited to, Pseudomonas aeruginosa; anaerobic sulfate-reducing bacteria, such as but not limited to, Desulfovibrio desulfuricans; and nontuberculous mycobacteria, such as but not limited to, Mycobacteria chelonei, fortuitum, and immunogenum. Examples of prominent fungal contaminants of MWF include, but are not limited to, Fusarium and Cephalosporium species. Among the yeasts, Candida and Trichosporon species are often isolated from contaminated MWF's.

To combat the negative affects of microbial growth on MWF's, it is important to monitor the MWF on a frequent basis. Therefore, MWF's should be tested periodically in order to detect specific microbe(s) growing therein, thus enabling the MWF to be treated with an appropriate biocide to control growth of the specific microbe(s) present therein.

The current methods of determining the condition of MWFs in order to respond to changes in the MWF have been inadequate to properly maintain the fluid. The common method of monitoring the properties of the MWF is to extract a sample of the fluid, remove it to a laboratory, and monitor the growth of microorganisms from the sample on various selective agar plates. Such procedures are generally regarded as defining the most critical parameter of the system, namely, the number of viable bacterial cells of a particular type in the MWF. However, these procedures have the disadvantage of requiring a great amount of excessive time for obtaining a result; for fungi, this type of testing can require one week, whereas for anaerobic bacteria and mycobacteria, which grow very slowly, determination of bacterial counts can take up to four weeks. Since these procedures are slow, and there is a delay between the time a sample is taken and the time at which results are obtained, changes in the MWF occur in many cases between sampling and analysis. Thus, the prior art methods of testing often produce data that are out of date due to the testing delay, or produce data that are inaccurate due to a change in the growth or viability of the microbes present subsequent to the drawing of the sample. All of these factors contribute to an increased uncertainty of the appropriate biocide to add to the MWF, as well as the correct time to add such biocide.

A number of approaches have been suggested to obtain this information more quickly. One such approach is to use PCR as a way to count the number of bacteria of various types in a system. Although this technique determines the number of genomes of a given bacterial type in a system, it gives no information at all about how many of these bacteria are viable. This is a fatal flaw, as decisions about biocide addition need to be based on the likelihood of future bacterial or fungal growth, rather than on a quantitation of the number of genomes present; such genomic data can be derived from various sources, including nonviable organisms and DNA-containing cellular debris, and is thus not an accurate indicator of viable microbes present in the sample.

Other potential approaches involve drying down a known volume of fluid onto a slide, staining with a specific stain, and then counting a known area. The number of bacteria that are seen can be related to the original volume, and a concentration can be determined. However, in addition to being very labor-intensive, this method also does not provide an indicator of the viability of the bacteria being counted.

The third major approach has been to use flow cytometry to enumerate specific populations of bacteria. Flow cytometry, broadly speaking, is a technique in which laser-based equipment is used to detect the presence and number of a particular cell or cells in a sample, and/or to determine one or more characteristics of cell(s) in a sample. There are several methods of staining cells for use in flow cytometric techniques—generally these methods are divided into (i) methods that use fluorescently derivatized molecules that interact with specific components of a bacterial cell membrane or cell wall (e.g., lectins that bind to carbohydrates, or antibodies against various surface antigens); and (ii) methods that use a fluorescent nucleic acid probe to bind to specific sections of RNA within permeabilized cells. Methods that depend on the use of surface-bound molecules as probes of species identity are susceptible to error in fluids that contain high levels of surface-active agents and high concentrations of dissolved metal salts, as MWF do. In addition, such methods are also susceptible to error when they are used to analyze non-cultured microbes, as cultured microbes are used to characterize the assay, and the surface antigens present on the noncultured microbes of the fluid composition may differ significantly in identity or accessibility to the fluorescent binding molecules when compared to the surface antigens on the cultured microbes used to characterize the assay. In contrast, nucleic acid-based methods detect components of the microbe that are highly invariant, regardless of the environment in which the microbe has been growing.

The use of viability stains on clinical Mycobacterial isolates has been published in several references. Also, the use of flow cytometry to quantitate mycobacteria in the absence of a viability determination has been described.

Presently, however, the art fails to teach or disclose methods for determining contamination that include both viability and species identification features. Therefore, a need exists in the art for improved methods and systems for detecting contamination in fluid compositions that overcome the disadvantages and defects of the prior art.

### SUMMARY OF THE INVENTION

The present invention is related to methods for determining contamination of a fluid composition. Broadly, the methods of the present invention include obtaining a sample of a fluid composition, such as a metalworking fluid, and separating any microbes present in the sample from the fluid composition of the sample, wherein the separation may be performed by centrifugation, filtration, dialysis, diafiltration, aggregation on a substrate, and/or combinations thereof. The microbes are then contacted with (i) an indicator adapted to differentiate between viable and nonviable microbes; and (ii) at least one species-specific or genus-specific probe. The microbes are then analyzed to determine viability based upon the indicator and to identify the species or genus of the microbes based upon the at least one species-specific or genus-specific probe. For example but not by way of limitation, a flow cytometry technique may be performed under conditions suitable to detect the indicator and the at least one species-specific or genus-specific probe.

In one embodiment, the method may further comprise the step of adding a known amount of at least one control microbe or control bead to the sample prior to the separating step to normalize for recovery in the subsequent steps.

The indicator utilized in accordance with the present invention may be an exclusion dye, and the indicator may permanently label the cells. In an alternative embodiment, the microbes may further be contacted with a second indicator that distinguishes viable microbes from cellular debris.

The at least one species-specific or genus-specific probe utilized in accordance with the present invention may include at least one mycobacterium specific probe and/or at least one probe specific for non-mycobacterium species. In one embodiment, the at least one species-specific or genus-specific probe may be a genetic probe, such as a peptide nucleic acid (PNA) probe, and the at least one species-specific or genus-specific probe may bind to a rRNA species of a species or genus of interest. Alternatively, the at least one species-specific or genus-specific probe may be a molecule that specifically binds to a second molecule present only on a surface of a target cell of the specific species or genus.

The at least one species-specific or genus-specific probe utilized in accordance with the present invention may be labeled with a fluorophore. The at least one species-specific or genus-specific probe may further include a quenching molecule, wherein the fluorophore is quenched when the species-specific or genus-specific probe is not bound to a target sequence. Alternatively, the fluorescent signal of the fluorophore may increase upon binding of the at least one species-specific or genus-specific probe to a target sequence.

In one embodiment of the present invention, the analysis may further include a determination of a metabolic state or environmental origin of a species or genus of microbe detected by the method.

In yet another embodiment of the present invention, at least two species-specific or genus-specific probes are utilized, and the step of analyzing further comprises the step of determining a metabolic state or environmental origin of the species or genus detected by the at least two probes.

The present invention also includes a method for performing quality control on a metalworking fluid, comprising performing the methods described herein above on a metalworking fluid composition at least twice during a quality control time period.

The present invention further includes a method for performing quality control services that includes obtaining a sample of a metalworking fluid composition from a user of the metalworking fluid composition; performing the methods described herein above on the sample to obtain contamination information about the sample; and communicating the contamination information to the user of the metalworking fluid composition, thus enabling the user to respond if one or more contaminants are present in the metal working fluid composition. This communication to the user may include additional information, such as but not limited to, (a) at least one biocide appropriate for killing a contaminant identified in the metalworking fluid composition to the user; (b) a current microbiological status of the system comprising a number of microbes and types of microbes of microbes present in the fluid composition and a fraction of the microbes that are viable; (c) trend analysis; (d) potential health concerns resulting from the test results; and (e) a recommended schedule for testing the fluid composition in the future. The present invention also includes a kit for analysis of a sample of fluid composition. The kit includes at least one viability stain as described herein above, and at least one species-specific or genus-specific labeled probe as described herein above. The kit may further include at least one of the following: (a) at least one sampling apparatus; (b) at least one separation apparatus for use in isolating microbes from a bulk sample; (c) at least one buffer for use with microbes; (d) a filtration apparatus; (e) an internal standard reagent; and (f) an external standard reagent.

Other objects, features and advantages of the present invention will become apparent from the following detailed description when read in conjunction with the accompanying figures and appended claims.

### BRIEF DESCRIPTION OF THE DRAWINGS

FIG. 1 is a flow diagram illustrating one embodiment of the method of the present invention.

FIG. 2 is a flow diagram illustrating another embodiment of the method of the present invention.

FIG. 3 is a flow diagram illustrating yet another embodiment of the method of the present invention.

FIG. 4 is a flow diagram illustrating yet another embodiment of the method of the present invention.

FIG. 5 is a flow diagram illustrating yet another embodiment of the method of the present invention.

FIG. 6 is a flow diagram illustrating yet another embodiment of the method of the present invention.

### DETAILED DESCRIPTION OF EXEMPLARY EMBODIMENTS OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients. All patents, patent applications, publications, and literature references cited in this specification are hereby incorporated herein by reference in their entirety.

As utilized in accordance with the present disclosure, the following terms, unless otherwise indicated, shall be understood to have the following meanings:

The term “fluid composition” as utilized herein will be understood to include metalworking fluids, but is not to be considered limited thereto. Rather, the term “fluid composition” as utilized in accordance with the present invention will also be understood to include any industrial fluid composition for which microbial contamination is a concern, such as but not limited to, industrial cleaning systems, industrial waste liquids, nuclear waste liquids, plating solutions, etching solutions, cooling tower systems, water treatment systems, fuel storage systems, refinery systems, crude oil well systems, municipal waste treatment systems and the like.

The terms “metal working fluid” and “MWF”, as used herein, will be understood to refer to a complex fluid composition that is applied to an interface between a tool and a metallic workpiece during the shaping of the workpiece by physical means, such as but not limited to, grinding, machining, turning, rolling, punching, extruding, spinning, drawing and ironing, pressing and drilling, stamping and forming, and the like. MWF can be broadly categorized as oils and aqueous based liquids or fluids. The oils are non-aqueous liquids comprising an oil or mixture of oils and one or more additives, such as for example, surfactants, extreme pressure agents, corrosion inhibitors, bactericides, fungicides and odor control agents. Aqueous based metalworking fluids are complex combinations of water, lubricant and additives. Aqueous based metalworking fluids can be classified as soluble oils, synthetic fluids and semi synthetic fluids.

The term “microbe” as used herein will be understood to refer to any prokaryotic, single-celled eukaryotic or protist organism, including but not limited to, bacteria, mycobacteria, fungi, and yeast. All the techniques described in this document can be adapted for the simultaneous detection, enumeration, and viability analysis of any single celled organism or group of organisms that are present in a homogenous or mixed population, as planktonic organisms or as part of a biofilm or biomass; as well as for the simultaneous detection, enumeration, and viability analysis of dissociated monomeric cells of a multicellular organism, including but not limited to: plants, algae, sponges, and animals that are present in a homogenous or mixed population.

The terms “nucleotide”, “oligonucleotide”, “polynucleotide”, “nucleotide segment” and “nucleic acid segment” referred to herein include deoxyribonucleotides and ribonucleotides, both naturally occurring and modified nucleotides. The term “modified nucleotides” referred to herein includes nucleotides with modified or substituted sugar groups and the like.

As used herein, the term “Peptide Nucleic Acid” or “PNA” refers to a chemical similar to DNA or RNA but differing in the composition of its “backbone.” PNA is an artificially synthesized oligomer. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by methylene carbonyl bonds. PNA's that can be utilized in accordance with the present invention are defined as any of the compounds referred to or claimed as Peptide Nucleic Acids in U.S. Pat. Nos. 5,539,082 or 5,623,049, or any compounds referred to as Peptide Nucleic Acids in published scientific literature, such as but not limited to, Diderichsen et al., Tett. Left. (1996) 37, 475-478; Fujii et al., Bioorg. Med. Chem. Left. (1997) 7, 637-640; Jordan et al., Bioorg. Med. Chem. Left. (1997) 7, 687-690; Krotz et al., Tett. Left. (1995) 36, 6941-6944; Lagriffoul et al., Bioorg. Med. Chem. Left. (1994) 4, 1081-1082; Lowe et al., J. Chem. Soc. Perkin Trans. 1, (1997) 1, 539-546; Lowe et al., J. Chem. Soc. Perkin Trans. 1, (1997) 1, 547-554; Lowe et al., J. Chem. Soc. Perkin Trans. 1, (1997) 1, 555-560; and Petersen et al., Bioorg. Med. Chem. Left. (1996) 6, 793-796. As used herein, the term “PNA oligomer” is defined as any oligomer comprising two or more PNA subunits (i.e., PNA residues or PNA monomers.

As used herein, the term “Locked Nucleic Acid” or “LNA” will be understood to refer to a modified RNA nucleotide. LNA is often referred to as inaccessible RNA. LNA is a synthetic nucleic acid analogue, incorporating “internally bridged” nucleoside analogues. The basic structural and functional characteristics of LNAs and related analogues are disclosed in various publications and patents, including WO 99/14226, WO 00/56748, WO 00/66604, WO 98/39352, U.S. Pat. No. 6,043,060, and U.S. Pat. No. 6,268,490, all of which are incorporated herein by reference in their entirety. See also, Braasch et al. (Chem. Biol. 8:17 (2001)), incorporated herein in its entirety by reference. Nielsen et al, (J. Chem. Soc. Perkin Trans. 1: 3423 (1997)); Koshkin et al, (Tetrahedron Letters 39:4381 (1998)); Singh & Wengel (Chem. Commun. 1247 (1998)); and Singh et al, (Chem. Commun. 455 (1998)). As with PNA, LNA exhibits greater thermal stability when paired with DNA, than do conventional DNA/DNA heteroduplexes. However, LNA can be synthesized on conventional nucleic acid synthesizing machines, whereas PNA cannot; special linkers are required to join PNA to DNA, when forming a single stranded PNA/DNA chimera. In contrast, LNA can simply be joined to DNA molecules by conventional techniques. LNA's may be obtained from Sigma Proligo (http://www.proligo.com/pro_primprobes/PP_{—}06-5.0_LNAOligos.html).

As used herein, the term “oligomer probe” will be understood to refer to any segment of nucleotide, oligonucleotide, polynucleotide, PNA oligomer or LNA oligomer suitable for hybridizing to a nucleic acid (DNA or RNA) sequence. The oligomer probe may be labeled with a detectable moiety or may be unlabeled.

The term “selectively hybridize” referred to herein means to detectably and specifically bind. Polynucleotides, oligonucleotides, LNAs, PNAs and fragments thereof in accordance with the invention selectively hybridize to nucleic acid segments under hybridization and wash conditions that minimize appreciable amounts of detectable binding to nonspecific nucleic acids. High stringency conditions can be used to achieve selective hybridization conditions as known in the art and discussed in more detail herein below. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence 5′-TATAC-3′ corresponds to a reference sequence 5′-TATAC-3′ and is complementary to a reference sequence 5′-GTATA-3′.

As used herein, the term “target sequence” is any defined nucleic acid sequence to be detected in an assay. The “target sequence” may comprise the entire sequence of interest or may be a subsequence of the nucleic acid target molecule of interest.

As used herein, the term “sensitivity” or “assay sensitivity” is defined as the difference in signal intensity caused by or attributable to the binding of detectable probe to its complementary sequence and any background or signal caused or attributable to any other source.

As used herein, the term “assay limit” or “limit of detection” is defined as the lower limit of signal intensity caused by the specific binding of detectable probe that can be detected above the background (noise).

As used herein, the terms “signal to noise” and “dynamic range” shall be interchangeable.

The terms “peptide” and “polypeptide” as used herein refer to molecules formed from the linking, in a defined order, of various α-amino acids. Such terms are generic terms to refer to native protein, fragments, or analogs of a peptide or polypeptide sequence.

The term “flow cytometry” as used herein refers to a technique for counting, examining and sorting microscopic particles suspended in a stream of fluid. Flow cytometry techniques allow simultaneous multiparametric analysis of the physical and/or chemical characteristics of single cells flowing through an optical/electronic detection apparatus. One type of flow cytometry that can be utilized in accordance with the present invention is “Fluorescence-activated cell-sorting” (FACS). FACS is a method for sorting a suspension of biologic cells into two or more containers, one cell at a time, based upon specific light scattering and fluorescent characteristics of each cell.

Regarding the terms “viable”, “viability”, “non-viable” and “non-viability”, there are many measures of viability in microbes, ranging from the ability to form colonies on appropriate agar to the presence of ATP in the microbial cytoplasm, or the ability to incorporate [³H]-thymidine into the cellular DNA. As used herein, the terms “viable” and “viability” will refer to microbes that have plasma membranes and/or cell walls that are impermeant to fluorescent molecules such as propidium iodide. This property correlates well with the ability of culturable microbes to form colonies on suitable culture medium. The terms “non-viable” and “non-viability” as used herein will refer to cells that have plasma membranes and/or cell walls that are permeable to fluorescent molecules such as propidium iodide. This property correlates well with an inability of culturable microbes to form colonies on suitable culture medium. The above-referenced techniques for measuring microbial viability are merely exemplary and are not meant to exclude or limit the techniques of measuring viability that may be utilized in accordance with the present invention; instead, any other measure of viability that corresponds well to the ability of microbes to survive in the fluids being tested may be utilized in accordance with the present invention.

The terms “fluorescent molecule” or “fluorophore” will be used interchangeably herein and will be understood to refer to any molecule or nanoparticle having the property of emitting light with a characteristic spectral distribution in response to being irradiated with light of an appropriate wavelength. It specifically includes quantum dots and similar nanoparticles as well as classical fluorophores. Regarding the selection of fluorophores utilized in accordance with the present invention, it is basic principle of assay design using fluorophores that the various fluorophores chosen should match one another appropriately. Among the important considerations are: (i) that all the fluorophores should have sufficient quantum yield to provide signal sufficient for detection using the apparatus at hand; (ii) that all the fluorophores should be excited with reasonable efficiency using the illumination sources available to the instrument being used to perform the assay; and (iii) that the fluorophores should have emission spectra with minimal mutual overlap, so that each fluorophore will be maximally distinguishable from the others being used in the assay (if more than one excitation wavelength is used, this will modify this consideration to the extent that fluorophores minimally excitable at one or the other wavelength need not be considered as overlapping a fluorophore substantially excited at the other wavelength, which would otherwise appear to have a significant spectral overlap). There are many other important considerations involved in the proper selection of fluorophores in these assays, which will be readily apparent to those skilled in the art of designing assays using fluorescent reporter molecules.

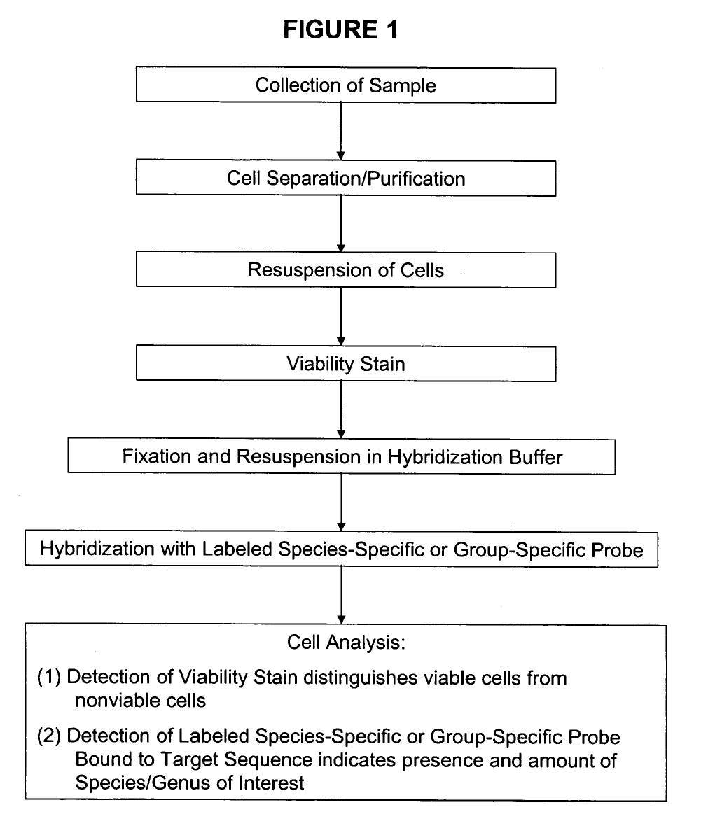

Shown in FIG. 1 is a flow diagram illustrating the steps of the methods of the present invention. Each of the steps is described herein in further detail herein below.

The first step of the methods of the present invention involves collection of a sample. Samples are collected by whatever means is most likely to obtain a sample that is representative of the system as a whole. Usually the samples utilized in accordance with the present invention are fluid samples, but this method is also compatible with samples of bacterial mats, biofilms, or residue. When a non-fluid sample is utilized, the sample will need to be dissociated and resuspended by an appropriate method before the rest of the process of the present invention can be performed.

The second step of the methods of the present invention involves an initial separation/purification of the cells of interest. In this step, microbes can be separated from the fluid by any cell separation method known in the art. These methods include, but are not limited to, centrifugation, filtration, dialysis, diafiltration, aggregation on any of a number of substrates, such as but not limited to, particles, beads derivatized with antibodies (such as but not limited to, monoclonal, polyclonal, single chain or of any other type of antibody, or any other molecule having an affinity for either a single or multiple populations of microbes within the fluid (such as but not limited to, lectins or Annexins) or having a surface with an intrinsic affinity for either a single or multiple populations of microbes within the fluid (for instance but not limited to, on the basis of charge or hydrophobicity)) that bind to surface antigens on all or a subset of the microbes present in the fluid, magnetic separation using derivatized magnetic beads or nanoparticles (the derivatization being the same as for nonmagnetic beads described herein previously), multivalent antibodies, and combinations thereof. It is within the ability of a person having ordinary skill in the art to determine which method is appropriate for a given circumstance. For most MWF, centrifugation, diafiltration, or aggregation are satisfactory.

In one embodiment, control microbes are added to the initial sample to normalize for recovery, if desired. Preferably, such control organisms may be labeled or otherwise modified to allow for their differentiation from microbes originally present in the sample. For example, but not by way of limitation, the control microbes could be genetically modified so as to express a fluorescent protein, such as but not limited to, green fluorescent protein (GFP); alternatively, the control microbes could be pre-stained with a persistent stain to allow their differentiation from microbes that are originally present in the fluid composition. In an alternative, calibrated fluorescent beads such as those available from Becton Dickinson (http://www.bd.com) or Bangs Laboratories (http://www.bangslabs.com/), may be utilized instead of the control microbes. However, such fluorescent beads may not behave in the same manner as an organism, and therefore may be an inferior normalization method; however, such method also falls within the scope of the present invention.

Generally, 2-10 ml of MWF will provide sufficient cells for analysis in the case of contamination; if a negative result is seen (provided that the control microbes or calibrated beads are present and therefore that the purification was successful), this will indicate that the fluid is acceptably free of microbial contamination. Typically, a preliminary centrifugation (such as but not limited to ≦500×g for 1-2 minutes), preliminary filtration through a wide mesh filter (such as but not limited to filtration through Whatman No. 1 paper) and/or an other preliminary separation technique is conducted to remove metal fines from the sample prior to the initial separation/purification step. If the cells are collected by centrifugation, 10 minutes at 13,000×g is generally sufficient to pellet them and allow the supernatant to be removed.

The third step of the methods of the present invention involves resuspension of the separated cells/microbes. Once the cells of interest are separated from the sample, the cells are resuspended in a buffer appropriate for staining and analysis. For example, but not by way of limitation, the cells may be resuspended in phosphate buffered saline (PBS). Generally, 2 ml of resuspension buffer will be an appropriate volume. Occasionally, if the microbes aggregate into tightly associated clumps, either intrinsically or as a result of the centrifugation, it is necessary to aggressively agitate the microbes to disaggregate them. Vortexing or sonication can be used for this purpose.

Any resuspension techniques known to a person having ordinary skill in the art can be utilized in accordance with the present invention, as long as such techniques do not affect the viability of the cells.

Once the cells of interest are resuspended in an appropriate buffer, a viability stain is performed. In this step, the microbes are stained with a differential stain for detection of viability of the cells. The viability stain utilized in accordance with the present invention may: (i) preferentially stain live/viable cells and not dead/non-viable cells; or (ii) preferentially stain dead/non-viable cells and not live/viable cells. The former type of viability stain is referred to as a dye uptake stain, where the dye/stain is normally taken up by viable cells but not taken up by non-viable cells. Any dye having such differential staining property can be used in accordance with the present invention to specifically label viable cells. Examples of dye uptake stains that may be utilized in accordance with the present invention include, for example but not by way of limitation, diacetylfluorescein, calcein AM, and carboxyfluorescein diacetate (CFDA). The use of CFDA as a dye uptake stain is disclosed in the following references: Sahar et al. (Cytometry, 15:213-221 (1994)) and Breeuwer et al. (Appl Environ Microbiol, 60:1467-1472 (1994)).

The latter type of viability stain is referred to as a dye exclusion stain (also referred to herein as an “exclusion dye”), where cells with an intact membrane are able to exclude the dye while cells without an intact membrane take up the coloring agent; the cell membranes and cell walls of “dead” cells have increased permeability to many molecules, and thus take up the differential stain, while the cell membranes and cell walls of “viable” cells maintain their ability to exclude foreign molecules. Any dye having such differential staining property can be used in accordance with the present invention to specifically label non-viable cells. For example, specific exclusion dyes that may be used in the methods of the present invention include, but are not limited to, any of the SYTOX® dyes from Molecular Probes (especially SYTOX® Green), Propidium Iodide (PI), 7-aminoactinomycin D (7-AAD), LDS 751, and ethidium monoazide.

In one embodiment, ethidium monoazide is utilized as the viability stain, as this exclusion dye has the additional advantage that it is reactive upon irradiation with light. Because of this, cells that are illuminated for several minutes after the ethidium monoazide has had a chance to bind to the accessible DNA (i.e., that of the dead cells) are labeled permanently. In a preferred embodiment, the indicator/stain (regardless of whether it is a dye uptake stain or an exclusion dye) will permanently label the cells. This property of permanent labeling will be shared by any molecule that has an affinity for DNA, and that is derivatized with a reactive group such as an azide; any newly prepared molecule with these properties could be used in this method and thus such molecules also fall within the scope of the present invention.

Other exclusion dyes that may be utilized in accordance with the present invention are dyes that are noncovalently associated with the DNA and thus can be washed out of the dead cells, or can re-equilibrate and label newly dead cells if the assay takes too much time.

The particular viability stains which are best suited for particular implementations of the method of the present invention depend on (i) the other dyes being used in the method, and (ii) the assay system utilized to detect the labeled microbes (so that spectral overlap of the emission profiles is minimized). For instance, in using commonly available FACS systems, it is advantageous to use dyes that can be excited at either 488 nm or 633 nm, as these are laser wavelengths generally available on these machines.

Following the viability stain, the next step of the method of the present invention involves fixation and resuspension of the cells of interest in hybridization buffer. After the nonviable microbial cells have been stained, the excess dye is removed by any method known in the art, such as but not limited to, filtration, dialysis, centrifugation, or chemical modification to a nonfluorescent molecule (such as but not limited to, reduction of NBD to ABD with sodium dithionite, as described by McIntyre et al. (Biochemistry, 30:11819-27 (1991)).

The cells are then resuspended at an appropriate concentration in new buffer. Following resuspension, the cells are then fixed and killed by any appropriate technique, such as but not limited to, addition of ethanol, and then permeabilized, such as but not limited to, by the addition of 200 μl of 4% paraformaldehyde for 60 minutes at room temperature. Following this, the cells are separated from the supernatant. This may be accomplished by centrifuging the solution and removing the supernatant, or subjecting the solution to filtration or dialysis. The cells are then resuspended in a hybridization buffer that is appropriate for the assay to be performed, as described in detail herein after.

In the next step of the method of the present invention, the cells are stained with a probe that is specific for a particular organism or group of organisms. This probe may be referred to as a microbe identification probe, and may be species-specific, genus-specific or group-specific. In one embodiment, this probe is a labeled, oligomeric probe that is designed so as to be complementary to a nucleic acid sequence in the genome of the organism or group of organisms that are to be quantified, but that is NOT complementary to an analogous nucleic acid sequence in other organisms or groups of organisms that may also be present in the sample. For example, if a probe were to be synthesized with the sequence CACTCCACCTCGCTT (SEQ ID NO:1), it would be precisely homologous to a portion of the 16s rRNA sequence as it exists in Mycobacterium fortuitum (GTGAGGTGGAGCGAA; (SEQ ID NO:2)), and be different from the analogous sequence in M. chelonei in only one of the 15 bases (GTAAGGTGGAGCGAA; (SEQ ID NO:3)), but differ in two bases relative to the analogous sequence in P. aeruginosa (GCGAGGTGGAGCTAA; (SEQ ID NO:4)). Because probes will bind to target sequences more tightly when they are more nearly precisely homologous, it is possible to perform the assay under conditions where the probe binds to this sequence in M. fortuitum and chelonei, but not in P. aeruginosa. Therefore, this offers a basis for discriminating between the two mycobacterial species on the one hand, and Pseudomonas on the other. It would also be possible to construct a probe that would bind only to the target sequence in Pseudomonas and not to that in either of the mycobacterial species. Finally, the sensitivity of the probe:target sequence binding to mismatches depends on the concentration of salt in the hybridization buffer. This allows for a given probe to be made more or less specific for sequences that match it precisely by varying the assay conditions. To continue with the example given above, by changing the buffer used to allow the probe to hybridize with its target sequence, one could force the probe to bind only to the sequence of M. fortuitum and not to either M. chelonei or P. aeruginosa. This means that by varying the assay conditions and/or the probe sequence, different assays can be conducted to enumerate either very specific species, or general groups of microbes, depending on the type of information needed. A number of factors are known that determine the specificity of binding or hybridization, such as pH; temperature; salt concentration; the presence of agents, such as formamide and dimethyl sulfoxide; the length of the segments that are hybridizing; and the like. There are various protocols for standard hybridization experiments. Depending on the relative similarity of the target DNA and the probe or query DNA, the hybridization is performed under stringent, moderate, or under low or less stringent conditions. For example, but not by way of limitation, in order to achieve labeling of Mycobacteria but not Pseudomonas, the following hybridization conditions can be utilized: a buffer of (10% w/v dextran sulfate, 30% v/v formamide, 0.1% w/v sodium pyrophosphate, 0.2% w/v polyvinyl-pyrrolidone, 0.2% w/v Ficoll, 1 mM disodium EDTA, 0.1% v/v Triton X-100, 10 mM Tris-HCl, adjusted to pH 7.5), and incubation at 56° C. for 2 hours with periodic gentle agitation. Other combinations of different buffers, incubation times, and incubation temperatures would also give these results. The probe and the hybridization conditions described herein are derived from those used in “Expeditious Identification and Quantification of Mycobacteria Species in Metalworking Fluids Using Peptide Nucleic Acid Probes” Skerlos et al., J Manuf Syst 22:136-147 (2003). The specificity of the hybridization assay can be tuned from genus-specific to species specific by increasing the stringency of the hybridization conditions by changing any of the several incubation reagent parameters; the ability to change the hybridization conditions in such a manner is within the ability of a person having ordinary skill in the art.

In a preferred embodiment, the microbe identification probe is a fluorescently labeled, oligomeric peptide nucleic acid (PNA) probe. The advantage of using a PNA probe in the methods of the present invention is that such PNA probe is not charged under the assay conditions, and thus it is able to move into the mycobacterial cytoplasm more efficiently than other known oligomeric probes. However, it is to be understood that any oligomeric probe capable of specific binding to a desired nucleic acid sequence in the genome of an organism or group of organisms can be utilized in accordance with the present invention. For example, but not by way of limitation, the oligomeric probe may be a DNA probe, an RNA probe, or an LNA probe.

In addition, the oligomeric probe can be labeled by any methods known in the art. Other examples of methods of labeling that could be utilized in accordance with the present invention include, but are not limited to, quantum dots and radioactivity. In one embodiment, ¹¹¹In could be utilized as the probe, and gamma ray Perturbed Angle Correlation Spectroscopy (PACS) could be utilized as the detection method, as described in more detail herein after. See for example, Hemmingsen et al. (Chem. Rev. 104:4027-4061 (2004)).

In one embodiment, the species-specific or genus-specific oligomeric probe is designed to bind to RNA within the cell. Because different genes in organisms are transcribed at different levels depending on the needs of the organism, it is best to choose a highly transcribed gene. One such set of genes are those that correspond to the ribosomal RNAs; these are needed for all transcription, and therefore many copies of this RNA species are present in most cells, under most conditions of life. This allows for more fluorescent oligomeric probe molecules to bind within each cell, and increases the fluorescent signal that is seen upon binding. This is one reason that ribosomal genes are particularly good targets for probes in assays of the type described herein and presently claimed. Another reason is that ribosomal genes are more highly conserved across species (i.e., they are more nearly similar across a greater range of species than many other genes). This allows a single probe to bind to target sequences of a greater number of related species (so, for example, it is easier to identify all Mycobacteria relative to other microbes such as Pseudomonas). A third reason is that the ribosomal RNA genes are often the first genes sequenced in newly discovered organisms; this means that more ribosomal RNA sequences are known than for many other genes.

While the above example describes oligomeric probes to rRNA target sequences, it is to be understood that the methods of the present invention are not limited to utilization of probes to such target sequences. The scope of the methods of the present invention includes the use of oligomeric probes that specifically bind to any target sequence present in a species or group of interest that can be utilized to distinguish between species or groups of interest. Such alternative target sequences include, but are not limited to, hrcA (a heat shock regulatory gene in gram positive bacteria), the recA gene, sigma(70) type factors, and the like (see, for example, http://tolweb.org/Eubacteria).

The species-specific or genus-specific oligomeric probes must be labeled in a manner that allows specific detection of the probe and that also does not destroy the ability of the probe to bind to its target sequence. In a preferred embodiment, the probe is labeled by derivatizing one end of the oligomer with a fluorescent molecule. This method of labeling retains the ability of the probe to recognize the target nucleic acid sequence. Two companies that prepare PNA oligonucleotides for such uses are: Bio-Synthesis, Inc (www.biosyn.com) and Applied Biosystems (www.appliedbiosystems.com).

Although any fluorophore that can be covalently linked to an oligomeric probe can be used in this application, there are several approaches to fluorescent labeling that are preferred in the method of the present invention. The principal limitation to simply labeling the oligomeric probe with a fluorophore is that the molecule will be fluorescent regardless of whether it is bound to a target RNA sequence or not. This means that unbound oligomers must be washed out of the microbial cells before they can be analyzed, or the background fluorescence is likely to be so high that it obscures the positive signal. Therefore, the methods of the present invention include alternatives that will reduce this background and allow for a high signal-to-noise ratio without adding a step to wash out unbound oligomeric probes.

In a first alternative, the oligomeric probe can be derivatized with a second molecule at the other end of the oligomer from the fluorophore. This second molecule must have the property of quenching the fluorescent emission signal of the fluorophore when it is physically in close proximity to the fluorophore. The efficiency of this quenching is proportional to the fourth power of the distance between the fluorophore and the quenching molecule; this means that quenching is very efficient when the two ends of the probe are close together, and very inefficient when they are farther apart. Because the oligomeric probe is able to bend in a fashion that allows the two ends of the oligomeric probe to come into close apposition when it is not bound to a target sequence, an oligomeric probe that is derivatized with a quenching molecule at the end opposite from the fluorophore will display a greatly reduced level of fluorescent emission at the characteristic emission maximum of the fluorophore when the oligomeric probe is in the unbound state. When the oligomeric probe binds to the target RNA sequence, it changes its conformation in a manner that increases the distance between the fluorophore and the quenching molecule (i.e., by having its whole length bound to the target RNA sequence, it is forced to straighten out so that the ends are now far apart). This allows the fluorophore to emit light efficiently at the characteristic emission maximum of the fluorophore only when it is bound to the target RNA sequence. Therefore, because an RNA molecule labeled in this fashion is only brightly fluorescent when it is bound to a proper RNA target sequence, it is not necessary to wash unbound oligomeric probe out of the cells before analysis. This makes the assay significantly simpler, quicker, and more effective.

There are two general classes of quenching molecules that can be used in accordance with the methods of the present invention. The first is another fluorescent molecule having an excitation spectrum that has substantial overlap with the emission spectrum of the fluorophore that is being used to label the oligomeric probe. The specific identity of the quenching fluorophore will depend on the identity of the fluorophore being used as a signal molecule, and thus any molecule known in the art that can function in such a manner will fall within the scope of the present invention. By way of example but not by way of limitation, a pair of fluorophore and fluorescent quencher that can be utilized in accordance with the present invention would be derivatizations using 4-chloro-7-nitrobenz-2-oxa-1,3-diazole (NBD) and rhodamine; many such potential pairs exist and the best such pair for a particular assay depends in part on the other fluorophores used in the assay. The second class of quenchers are molecules that have absorbance spectra that substantially overlaps with the emission spectrum of the fluorophore that is being used to label the oligomeric probe, but which themselves are not fluorescent. Examples of this class of molecules include the “Black Hole Quencher” group of molecules (developed by BioSearch Technologies (www.biosearchtech.com)), and the “Eclipse Dark Quencher” molecule (developed by Epoch Biosciences (www.epochbio.com)). In addition, there are other such nonfluorescent molecules available that will be known to a person having ordinary skill in the art that can be utilized in accordance with the present invention.

In a second alternative, a preferred method of increasing the signal-to-noise ratio of bound versus unbound oligomeric probe is to use a fluorophore that increases its fluorescent signal after the probe binds to the target sequence. This is a property of some fluorophores whose quantum yield is dependent on the local environment of the fluorophore. When the environment of the fluorophore changes in an appropriate way after binding of the oligomeric probe to the target sequence, it can be detected in one of three ways: (i) the fluorescent signal at the maximal emission wavelength will increase substantially after the probe binds to the target sequence; (ii) the maximal emission wavelength will change after the probe binds to the target sequence; or (iii) a combination of (i) and (ii). Any fluorescent dye having this property when conjugated to oligonucleotides (in some cases to PNA oligonucleotides) may be utilized in accordance with the present invention. Examples include, but are not limited to, thiazole orange (TO), and the SYBR 101, 102, and 103 fluorophores available from Molecular Probes (www.probes.com).

Because it is possible to detect changes in the fluorescent signal that are caused by even a single basepair mismatch between the oligomeric probe and the target sequence, this method can also be used to detect and enumerate multiple species at a single time (by separating the signal for organisms with a perfect sequence match between the probe and the target sequence from the signal for organisms having various degrees of mismatch between the sequence of the probe and the target sequence). References that discuss this phenomenon include Svanvik et al. (“Detection of PCR products in real time using light-up probes.” Anal Biochem, 287:179-182 (2000)), and Svanvik et al. (“Light-up probes: thiazole orange-conjugated peptide nucleic acid for detection of target nucleic acid in homogeneous solution.” Anal Biochem, 281:26-35 (2000)). Therefore, the detection of multiple species using a single oligomeric probe in a single hybridization analysis step falls within the scope of the methods of the present invention.

In order to analyze the samples prepared by the methods of the present invention, it is necessary to identify which microbial cells are associated with which fluorescent signals (which in turn correspond to the various identity parameters being tested). The simplest methods to achieve this end involve resolving the population of microbial cells one at a time, by (i) illuminating them with incident radiation at a wavelength appropriate to excite the fluorophores being used as labels in the assay (this may involve sequential excitation at more than one wavelength), and (ii) detecting the emitted radiation that comes from the various fluorophores that may be associated with the microbial particle, depending on its identity and consequent association with the various probes being used in the assay (e.g., a nucleic acid stain, or a fluorescently labeled PNA oligonucleotide). One analytical instrument that is able to accomplish this task is a flow cytometer—a Fluorescence-Activated Cell Sorter (FACS) instrument. Instruments of this sort are made by Becton Dickinson (www.bd.com) as well as several other companies, and include the FACSCalibur™ and the FACSCanto™. The precise details of defining the parameters by which one population of microbes is differentiated from another are complex and depend strongly on the precise identity of the fluorophores being used, the nature of the microbes being enumerated, the original fluid in which they grew, and the instrument itself. Therefore, these details will not be specified but nevertheless are the result of choices that are obvious to those skilled in the art and science of assay design for flow cytometric applications.

Although flow cytometers are one class of instrument that is able to analyze samples prepared according to the methods described herein, there are a number of other instruments that can achieve the same result by different methods. Therefore, the present invention is not limited to the use of a flow cytometer, and thus other instruments capable of cell analysis as described herein also fall within the scope of the present invention. Examples of other methods of detection analysis that are also within the scope of the present invention include, but are not limited to, placement of the microbes onto a filter (by filtration, diafiltration, or some other method) followed by illumination through laser scanning and detection; fluorescence microscopy; surface plasmon resonance-based methods; and methods which use nanoshells of the sort made by NanoSpectra BioSciences (www.nanospectra.com).

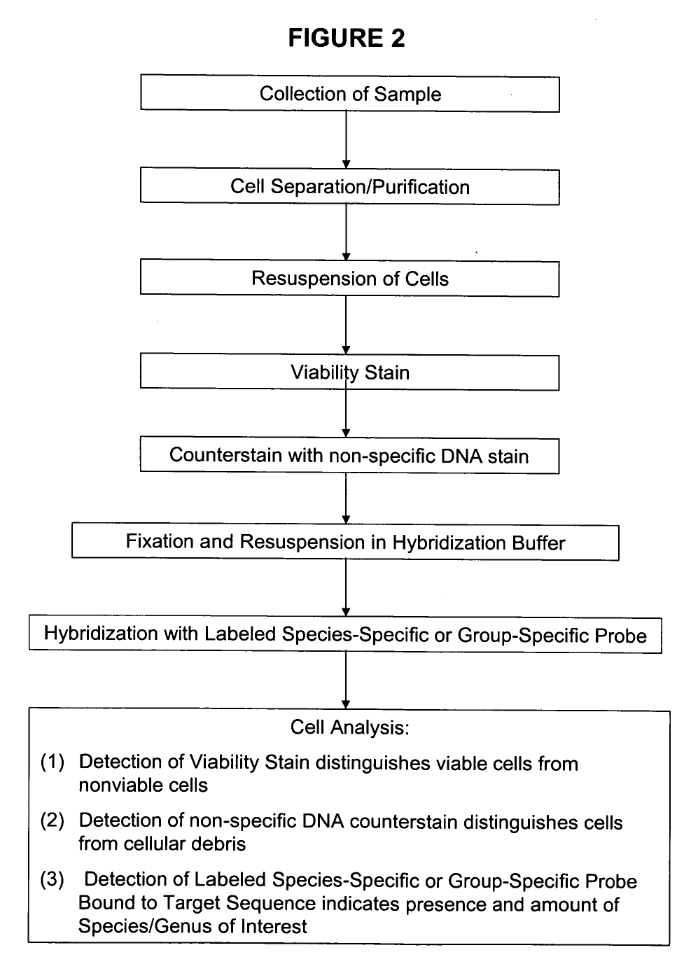

In one embodiment of the method of the present invention, the method further comprises the step of counterstaining with a nonspecific DNA stain, as shown in the flow diagram of FIG. 2. Many of the particles seen in field samples are not actually whole microbes but instead represent cellular debris and small particles of unknown origin. It is therefore desirable to exclude such contamination from being included in the enumeration of actual microbes. One distinguishing property of microbes in general is that they contain DNA. Therefore, debris can be distinguished from microbes by using a fluorescent stain that binds to the DNA of all cells, whether they are viable or not. Many such stains exist; examples include the SYTO® dyes available from Molecular Probes (www.probes.com). Because these dyes are membrane-permeable, they are more desirable in this application than the SYTOX® dyes which will only stain cells with permeabilized membranes. This property allows the SYTO® family of dyes to be used in both the assay schemes described above as well as in alternatives thereof described in further detail herein below. Although this step is useful in increasing the signal-to-noise ratio and giving more accurate results, it is optional and can be eliminated.

While FIG. 2 illustrates this counterstaining step as occurring after the viability stain step, it is to be understood that the counterstaining step may occur at any other point during the method of the present invention, including but not limited to, after the hybridization step. In addition, the counterstaining step could be performed in combination with the viability staining step.

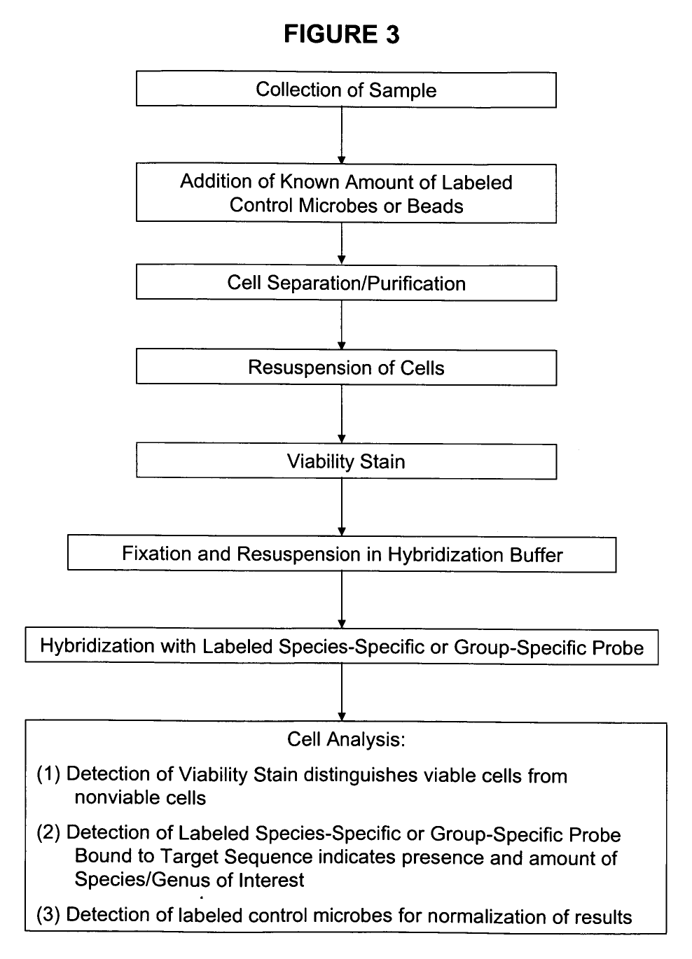

In an alternative embodiment of the methods of the present invention, the ability to account for the fraction of microbes that are lost during the purification and labeling process, and thus normalize the results obtained, is provided. This normalization of results is accomplished by adding a control population of a known number of prelabeled microbes to the collected sample prior to the cell separation step (see FIG. 3). These microbes should correspond to the population of interest; for example, if mycobacteria are to be enumerated by the assay, a known number of control mycobacteria are added to the sample. If multiple microbial types are to be enumerated, one can either (i) add all the relevant populations at once, or (ii) add them to separate aliquots of the sample and perform multiple labelings and assays. Option (i) has the advantage of only requiring one isolation and labeling step; option (ii) allows each experiment to be done with fewer simultaneous fluorescent dyes, and therefore can be performed on a simpler instrument. The control microbes should be labeled before being added to the assay using a label that is (i) unique within the assay in its excitation and emission spectra; (ii) persistent within the control microbe population; and (iii) nontransferrable so that it does not begin to label the experimental microbes as well. One example of a dye that will fit this profile is Green Fluorescent Protein (GFP) or a related fluorescent protein expressed within the microbe; however, any label that meets the requirements listed above may be utilized in accordance with the present invention.

While the use of control microbes have been described herein previously, it is to be understood that fluorescently labeled beads may also be utilized as internal standards in accordance with the present invention. However, the use of control microbes over labeled beads is preferred in the methods of the present invention, as the beads could potentially behave differently during the purification, introducing an error into the recovery calculation.

Although this approach will increase the accuracy of the results, it is important to know that even results with imprecise accuracy are often sufficient to be useful in diagnosis and maintenance of these systems, in accordance with the present invention. Indeed, in many industrial applications, knowing the number of various classes of microbes to within 1 to 2 logs of concentration (a 10-100 fold concentration range) is useful and worthwhile.

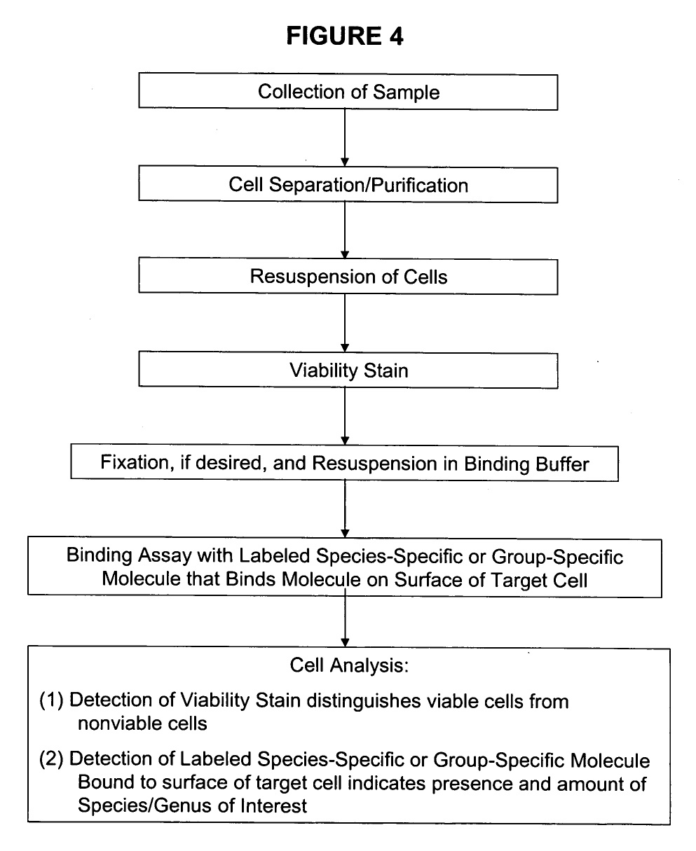

In yet another alternative embodiment of the method of the present invention, a binding assay with a fluorescently labeled molecule that species-specifically or genus-specifically binds to a target cell may be utilized rather than, or in combination with, the step of hybridization with a labeled species-specific or genus-specific probe (see FIG. 4). The molecule utilized in the binding assay of the methods of the present invention may be any molecule which can be fluorescently labeled and which has the additional property of binding to a second molecule that exists only on the surface of a desired target cell. Included in this embodiment are labeling strategies that utilize an unlabeled primary antibody or other binding molecule that binds the target molecule on the target cell and a labeled secondary antibody or other binding molecule that binds the primary antibody or other molecule. The particular assay conditions for this assay will depend on the antibody utilized and the accessibility of the antigen on the target cell; however, such conditions will be apparent to a person having ordinary skill in the art. General assay conditions will include 0.1% BSA in PBS, along with a surfactant such as but not limited to, an octylglucoside.

In one embodiment, such molecule may be an antibody that specifically binds to lipoarabinomannan (LAM) or lipomannan (LM), members of a family of lipoglycans that are found on the surface of various species of mycobacteria, in a species-specific distribution. See, for example, Briken et al. (“Mycobacterial lipoarabinomannan and related lipoglycans: from biogenesis to modulation of the immune response.” Mol Microbiol. 53:391-403 (2004)). These antibodies can be made by any currently available or future method of creating antibodies (either using whole animals such as mice, rabbits, or chickens, or molecular genetic methods such as phage display, or combinations of these methods). They will be fluorescently labeled using fluorophores that appropriately fit with the emission and excitation spectra of the other fluorophores used in the assay.

In addition to the antibody example given above, it is to be understood that any molecule that binds specifically with a molecule found only (or predominantly) on the surface of a species or group of microbes that are to be identified can be fluorescently labeled and used as a probe in accordance with the method of the present invention. Examples of such molecules include, but are not limited to, lectins. Lectins are molecules that bind specifically to various sugar groups on surfaces. If a microbe has a unique sugar, it will be specifically bound by a lectin that binds to that sugar. An example of a lectin that may be utilized in accordance with the present invention includes, but is not limited to, DC-SIGN. LAM (lipoarabinomannan) is a glycolipid found (probably) only on mycobacteria, and it is a ligand for DC-SIGN.

Regarding the example method illustrated in FIG. 4, the method may further comprise a fixation step prior to the binding assay. While fixation when using a binding assay for detection of the microbe identification probe is optional, a fixation step is preferred, as it opens up more sites for binding and reduces any safety concerns posed by certain microbes that may be present in the sample.

If the assay uses a fluorescent probe to identify the microbe being enumerated that does not require the microbe to be permeabilized for labeling, it is possible to use a number of fluorescent viability stains that are minimally compatible with the microbe identification probe. These include molecules which have the properties of (i) not being fluorescent themselves, (ii) being able to cross microbial cell walls and cell membranes, (iii) being substrates for intracellular enzymatic processes such as esterases, (iv) having one of the molecular products of such a reaction being both fluorescent and not able to cross microbial cell walls and cell membranes. Molecules having this set of properties will fluorescently label the interior of cells which are viable. Examples of such molecules include calcein AM, BCECF (2′,7′-bis-(2-carboxyethyl)-5-(and-6)-carboxyfluorescein, acetoxymethyl ester) AM, and CellTracker™ Green CMFDA (5-chloromethylfluorescein diacetate), all available from Molecular Probes (www.probes.com).

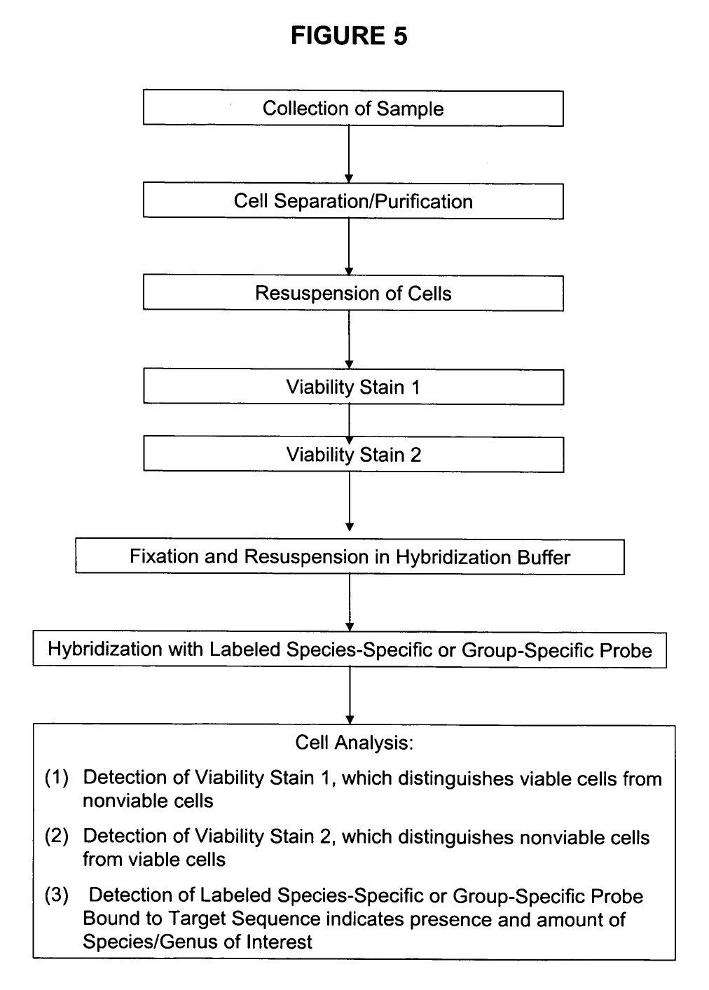

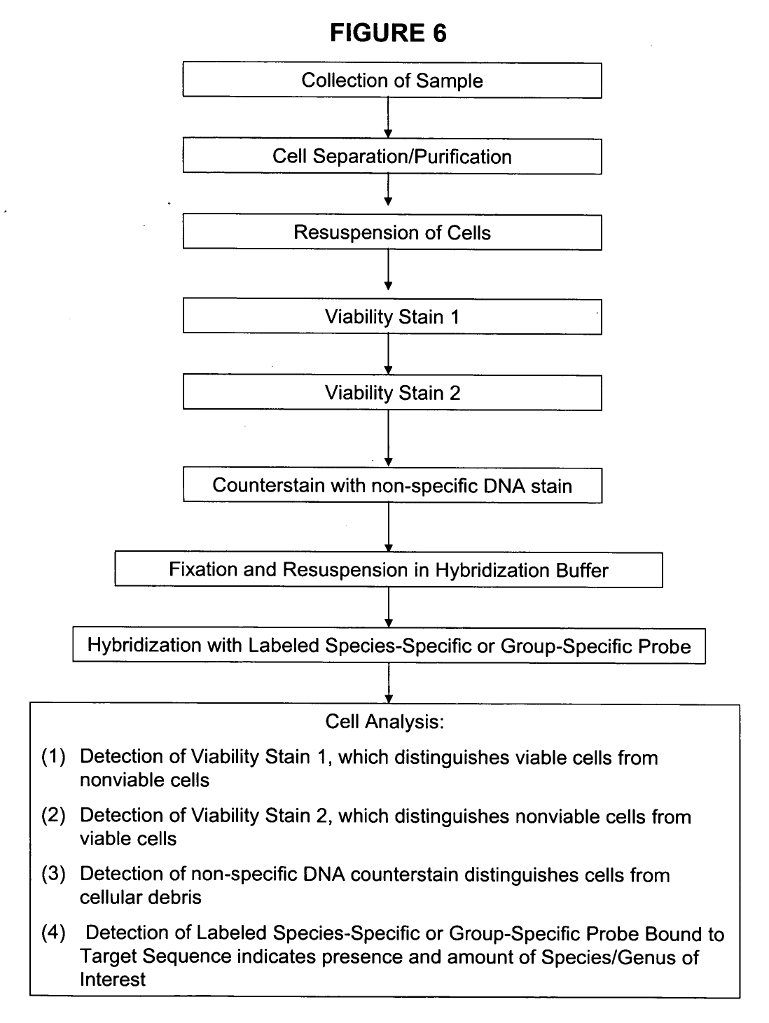

The labeling of viable cells with a fluorophore and the labeling of a specific group of microbes (e.g., the mycobacteria) with another fluorophore, can be combined with the labeling of nonviable cells with a third fluorophore as discussed above if desired (see FIG. 5). Finally, the cells can be labeled with a fourth fluorophore that stains all DNA as discussed above in order to ensure that positive events are confined to those representing actual microbes (see FIG. 6). Any and/or all of the viability/counterstains illustrated in FIGS. 5 and 6 may be conducted in a single step/assay, or may be conducted in separate assay steps, depending on the assay conditions utilized for each stain.

In addition, the binding assay described herein above may be substituted for the hybridization assay in any method described herein, and thus the use of the binding assay with more than one viability stain and/or counterstain falls within the scope of the present invention. Thus, the present invention is not strictly limited to the individual flows of steps shown in FIGS. 1-6.

In yet another embodiment of the methods of the present invention, assays are provided that will allow determination of the metabolic state or environmental origin of the microbe. The fact that the oligomeric probes described herein can be designed to bind to RNA within the cell can be used to create assays where additional target genes are selected to give information about the metabolic state and prior environment of the microbe. The transcriptional levels of many genes are regulated by the microbe in response to its environment. For example, when microbes exist in a biofilm, they completely change the pattern of expression of their genes relative to when they are growing as planktonic bacteria free in a fluid. In this condition of growth, many genes have increased levels of expression, while others have decreased levels of expression. By choosing two appropriate genes, one with an increased expression level and one with a decreased expression level in response to growth in a biofilm, it is possible to know whether a given microbe has recently been growing in a biofilm, or whether it has been planktonic for some time. In such an application, each of the oligomeric probes will be labeled with a different fluorophore, each having distinct spectral properties that will allow it to be distinguished uniquely within the assay system. The amount of fluorescent staining seen in response to each of the probes will be proportional to the expression of that gene in an individual microbe. The ratio of the expression levels of the two target genes will indicate the environmental origin of the microbe. Ideally, a third oligomeric probe (labeled with a third fluorophore) would also be used to identify the species of microbe. The specific genes chosen will depend on microbial species (or multiple species) being targeted, as different genes are regulated differently in various species. Identification of microbes that have recently been growing in a biofilm is only a representative example of this general class of assays, and the scope of the present invention includes any method that detects changes in gene expression as a result of microbial growth environment for determining the metabolic and/or growth state of the target microbes.

The present invention also provides methods for performing quality control on a metalworking fluid composition. Such methods comprise performing the methods described herein on a metalworking fluid composition at least twice during a quality control time period.

In one embodiment, a sample of metalworking fluid composition is obtained from a user of the metalworking fluid composition, and one or more of the methods described herein above are utilized to obtain contamination information about the sample of metalworking fluid composition. The contamination information is then communicated to the user of the metalworking fluid composition, thus enabling the user to respond to one or more contaminants present in the metalworking fluid composition by application of an appropriate biocide. In one embodiment, the method may further include conveying information concerning at least one biocide appropriate for killing a contaminant identified in the metalworking fluid composition to the user. In addition, other information may be conveyed to the user, such as but not limited to, the current microbiological status of the system (number and types of microbes, fraction that are viable), trend analysis (in combination with previous results from the same system), potential health concerns resulting from the test results, how often the system should be tested in the near future, and the like.

The present invention also provides for a kit comprising a viability stain and a species-specific or group-specific probe for utilization in the methods of the present invention. The kit may further include reagents for purification of microbes away from the bulk fluid (i.e., separation means), and buffers for use in at least one of the resuspension steps and for use in the hybridization and/or binding assays.

Eventually, it is likely that flow cytometers will become cheap and simple enough to be ubiquitous in industrial fluid testing laboratories. Therefore, a kit in accordance with the present invention for use in such laboratories would be extremely beneficial. Such a kit may include, in accordance with the present invention, one or more of the following: (i) at least one sampling apparatus designed to obtain a representative sample with minimal contamination from unemulsified tramp oil, wherein such sampling apparatus may be disposable (several different sampling apparatuses may be created, optimized to handle different sorts of samples, such as but not limited to, the bulk fluid, tramp oil itself, residue, biofilms, or aerosols); (ii) at least one separation apparatus for use in isolating microbes from the bulk sample (for example, but not by way of limitation, filtration apparatus, tubes for centrifugation, reagents such as derivatized beads to aggregate the microbes, derivatized magnetic beads, magnetic separation apparatus, and the like); (iii) resuspension buffers for use with the microbes after they are isolated; (iv) viability stains (uptake and/or exclusion dyes); (v) nonspecific DNA stains; (vi) filtration apparatus for removing microbes from the resuspension/staining buffer; (vii) fixation and hybridization buffers for the species-specific or genus-specific probe; (viii) one or more species-specific or genus-specific probes, whether PNA, LNA, DNA, RNA, or some other oligomer, labeled either solely with a fluorophore or with a fluorophore in combination with a quencher or RET acceptor; (ix) assay buffer for performing the analysis assay (which could be in a flow cytometer or any of the other instruments described); (x) any other fluids required to perform the assay, such as sheath fluid; (y) an internal standard reagent, such as but not limited to, fluorescent beads or labeled control microbes, as described herein; and (z) an external standard reagent such as but not limited to, known numbers of labeled, fixed microbes of known species distribution.

An Example is provided hereinbelow. However, the present invention is to be understood to not be limited in its application to the specific experimentation, results and laboratory procedures. Rather, the Example is simply provided as one of various embodiments and is meant to be exemplary, not exhaustive.

### EXAMPLE

2-10 ml of metalworking fluid composition was collected as a sample, filtered through Whatman No. 1 filter paper to remove metal fines, and subjected to centrifugation at 13,000×g for 10 minutes to pellet cells present in the sample. The supernatant was removed, and the cells were resuspended in 2 ml PBS using vortexing or slight sonication so as not to damage the cells.

The resuspended cells were then incubated with the viability stain SYTOX® Red at 3 μM concentration (stains dead cells) and CFDA at 30 μM concentration (stains live cells) for 15 minutes at room temperature in the dark. Following incubation, the excess dye was removed by centrifugation at 13,000×g for 10 minutes, washed once in 500 μl PBS, and recentrifuged at 13,000×g for 10 minutes. The cells were then resuspended in 700 μl PBS.

Following resuspension, the cells in the solution were fixed and killed with 500 μl ethanol and permeabilized by the addition of 200μ of 4% paraformaldehyde for 60 minutes at room temperature. The solution was then centrifuged at 13,000×g for 10 minutes, the supernatant removed, and the cells resuspended in 200 μl of hybridization buffer comprising 10% w/v dextran sulfate, 30% v/v formamide, 0.1% w/v sodium pyrophosphate, 0.2% w/v polyvinyl-pyrrolidone, 0.2% w/v Ficoll, 1 mM disodium EDTA, 0.1% v/v Triton X-100, 10 mM Tris-HCl, adjusted to pH 7.5.

The mycobacterial-specific probe of SEQ ID NO:1 was purchased from Bio-Synthesis (www.biosyn.com). The PNA was labeled with 4-chloro-7-nitrobenz-2-oxa-1,3-diazole (NBD) and the BlackHole Quencher DHQ-1 from BioSearch Technologies using standard methods.

10 μl of the labeled probe (at 1 nM final concentration) of SEQ ID NO:1 was added to the cells resuspended in the hybridization buffer, and the hybridization reaction involved incubation at 56° C. for 2 hours with periodic gentle agitation. No wash steps were required due to the presence of the quenching molecules.

The solution is then subjected to flow cytometry using a Becton Dickinson FACSCanto™ fluorometer. CFDA has an emission maximum at 513 nm, and NBD has an emission maximum at 540 nm; these are detected using the 488 nm laser (CFDA with a 515/10 filter and NBD with a 550/20 filter). The SYTOX® Red has an emission maximum at 658 nm and is detected using the 633 nm laser (with a 660/20 filter). Corrections for crossover of dyes into neighboring fluorescence channels were obtained using standard methods. FSC and SSC signals were also obtained and were used to eliminate false signals due to cellular debris and other particles.

Events were gated in accordance with signals obtained using labeled control samples of known bacteria; in all cases at least 100,000 events scored as positively a microbe were counted to generate results.

Thus, in accordance with the present invention, there has been provided methods for determining contamination of a fluid composition that fully satisfies the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

1. A method for determining contamination of a fluid composition, comprising the steps of:
obtaining a sample of a fluid composition;
separating any microbes present in the sample from the fluid composition of the sample;
contacting the microbes with an indicator adapted to differentiate between viable and nonviable microbes;
contacting the microbes with at least one species-specific or genus-specific probe; and
analyzing the microbes to determine viability based upon the indicator and to identify the species or genus of the microbes based upon the at least one species-specific or genus-specific probe.

2. The method according to claim 1 wherein, in the step of obtaining a sample of a fluid composition, said fluid composition is a metalworking fluid.

3. The method according to claim 1 wherein, in the step of separating any microbes present in the sample from the fluid composition of the sample, the separation is performed by a method selected from the group consisting of centrifugation, filtration, dialysis, diafiltration, aggregation on a substrate, and combinations thereof.

4. The method according to claim 1 further comprising the step of adding a known amount of at least one control microbe or control bead to the sample prior to the separating step to normalize for recovery in the separating step.

5. The method according to claim 1 wherein, in the step of contacting the microbes with an indicator, the indicator comprises an exclusion dye.

6. The method according to claim 1, wherein the step of contacting the microbes with an indicator further comprises contacting the microbes with a second indicator that distinguishes viable microbes from cellular debris.

7. The method according to claim 1 wherein, in the step of contacting the microbes with an indicator, the indicator permanently labels the microbes.

8. The method according to claim 1 wherein, in the step of contacting the microbes with at least one species-specific or genus-specific probe, the at least one species-specific or genus-specific probe includes at least one mycobacterium specific probe.

9. The method according to claim 8, wherein the at least one species-specific or genus-specific probe further comprises at least one probe specific for non-mycobacterium species.

10. The method according to claim 1 wherein, in the step of contacting the microbes with at least one species-specific or genus-specific probe, the at least one species-specific or genus-specific probe is a genetic probe.

11. The method according to claim 10, wherein the at least one species-specific or genus-specific probe is a peptide nucleic acid (PNA) probe.

12. The method according to claim 10, wherein the at least one species-specific or genus-specific probe binds to a rRNA species of a species or genus of interest.

13. The method according to claim 1 wherein, in the step of contacting the microbes with at least one species-specific or genus-specific probe, the at least one species-specific or genus-specific probe is a molecule that specifically binds to a second molecule present only on a surface of a target cell of the specific species or genus.

14. The method according to claim 1 wherein, in the step of contacting the microbes with at least one species-specific or genus-specific probe, the at least one species-specific or genus-specific probe is labeled with a fluorophore.

15. The method according to claim 14, wherein the at least one species-specific or genus-specific probe also comprises a quenching molecule, wherein the fluorophore is quenched when the species-specific or genus-specific probe is not bound to a target sequence.

16. The method according to claim 14, wherein the fluorescent signal of the fluorophore increases upon binding of the at least one species-specific or genus-specific probe to a target sequence.

17. The method according to claim 1, wherein the step of analyzing comprises conducting a flow cytometry technique under conditions suitable to detect the indicator and the at least one species-specific or genus-specific probe.

18. The method according to claim 1, wherein the step of analyzing further includes determining a metabolic state or environmental origin of a species or genus of microbe detected by the method.

19. The method according to claim 1 wherein, in the step of contacting the microbes with at least one species-specific or genus-specific probe, at least two species-specific or genus-specific probes are utilized, and the step of analyzing further comprises the step of determining a metabolic state or environmental origin of the species or genus detected by the at least two probes.

20. A method for performing quality control on a metalworking fluid, comprising performing the method of claim 1 on a metalworking fluid composition at least twice during a quality control time period.

21-33. (canceled)


SEQUENCE LISTING (4 sequences)

SEQ ID NO: 1 - DNA, 15 bp, Mycobacterium fortuitum
cactccacctcgctt

SEQ ID NO: 2 - DNA, 15 bp, Mycobacterium fortuitum
gtgaggtggagcgaa

SEQ ID NO: 3 - DNA, 15 bp, Mycobacterium chelonei
gtaaggtggagcgaa

SEQ ID NO: 4 - DNA, 15 bp, Pseudomonas aeruginosa
gcgaggtggagctaa